(19) United States
(12) Patent Application Publication
(10) Pub. No.: US 2022/0175862 A1
(43) Pub. Date: Jun. 9, 2022

(54) CO-ADMINISTRATION COMPOSITION FOR PREVENTING OR TREATING HYPERCHOLESTEROLEMIA, CONTAINING BALLOON-FLOWER EXTRACT OR PLATYCODIN D DERIVED THEREFROM AS ACTIVE INGREDIENT

(71) Applicant: University-Industry Cooperation Group of Kyung Hee University, Gyeonggi-do (KR)

(72) Inventor: Seong-Gyu Ko, Seoul (KR)

(21) Appl. No.: 17/427,557

(22) PCT Filed: Feb. 3, 2020
(86) PCT No.: PCT/KR2020/001584

§ 371 Date: Feb. 28, 2022

(30) Foreign Application Priority Data
Feb. 1, 2019 (KR) 10-2019-0013726

(51) Int. Cl. A61K 36/346; A61K 31/366; A61K 45/06

(52) U.S. Cl. CPC A61K 36/346; A61K 45/06; A61K 31/366

(57) ABSTRACT

Provided are a pharmaceutical composition and a method for preventing or treating hypercholesterolemia, the pharmaceutical composition including a Platycodon grandiflorum extract or Platycodin D derived therefrom, and a statin-class drug as active ingredients. Also disclosed is a method of increasing expression of low-density lipoprotein receptor, the method including the step of treating hepatocytes in vitro with a Platycodon grandiflorum extract or Platycodin D derived therefrom.

### BACKGROUND OF THE INVENTION

### 1. Field of the Invention

The present disclosure relates to a composition for combined administration, the composition including a Platycodon grandiflorum extract or Platycodin D derived therefrom, and specifically a pharmaceutical composition for combined administration for preventing or treating hypercholesterolemia, the pharmaceutical composition including a Platycodon grandiflorum extract or Platycodin D derived therefrom, and a statin-class drug as active ingredients; an anti-hypercholesterolemia supplement agent including a Platycodon grandiflorum extract or Platycodin D derived therefrom as an active ingredient; a kit for preventing or treating hypercholesterolemia, the kit including a Platycodon grandiflorum extract or Platycodin D derived therefrom, and a statin-class drug as active ingredients; a method of preventing or treating hypercholesterolemia, the method including the step of administering, to an individual excluding humans, a Platycodon grandiflorum extract or Platycodin D derived therefrom, and a statin-class drug in combination; a composition for treating or preventing hypercholesterolemia, the composition including a Platycodon grandiflorum extract or Platycodin D derived therefrom as an active ingredient; and a method of increasing expression of low-density lipoprotein receptor, the method including the step of treating hepatocytes in vitro with a Platycodon grandiflorum extract or Platycodin D derived therefrom.

### 2. Description of the Related Art

Cholesterol is a principal component of cell membranes and lipoproteins, and as a precursor of bile acids, steroid hormones, and vitamin D3, it is synthesized in most tissues except brain tissue and most actively in the liver. Most cholesterol in the body is made by the liver and delivered to other organs through forms of low-density lipoprotein (LDL) particles that package cholesterol. The liver removes plasma LDL-C via hepatic low-density lipoprotein receptor (LDLR), which is a cell surface protein binding to LDL, and mediates uptake of LDL particles into cells via endocytosis.

Recently, cholesterol-related diseases are occurring in many modern people due to Western-type diets. Excess plasma cholesterol that is not utilized in or excreted from the body is known as a risk factor for diseases of the cardiovascular system, such as hypercholesterolemia and arteriosclerosis.

For patients with these diseases, chemotherapy has been widely used. Representative chemotherapeutic agents that have been used for hypercholesterolemia include statin-class drugs, such as lovastatin, simvastatin, pravastatin, fluvastatin, etc. Chemotherapy is effective for some diseases, but it causes side effects in patients when continuously administered in large doses. Moreover, current chemotherapeutic agents are known to have numerous adverse side effects due to their non-specific cytotoxicity. Specifically, chemotherapeutic agents affect not only abnormal cells but also normal cells, and are reported to cause diabetes, muscle pain, lethargy, cognitive decline, sexual dysfunction, cataracts, insomnia, weakened immunity, gingival necrosis, aggressive behavior, etc., although these vary depending on drugs. As a method of reducing these side effects, there is a method of administering a natural extract, which has been proved to be safe in the body, in combination with a chemotherapeutic agent. It is known that not only can this combined therapy prevent continuous administration of a large dose of the chemotherapeutic agent to patients, but it can also enhance the effect of the chemotherapeutic agent itself.

Meanwhile, Platycodon grandiflorum (PG), commonly known as the balloon flower, is a medicinal herb whose natural extract is used in numerous applications. Platycodon grandiflorum (PG) is, also called Platycodon grandiflorum root, a medicinal herb prepared by removing the root or periderm of the balloon flower belonging to the family Campanulaceae. Further, Platycodin D (PD) isolated from Platycodon grandiflorum is known to regulate numerous biological processes involved in apoptosis, inflammation, oxidative stress, and hepatotoxicity. It has also been reported that Platycodin D may be usefully applied to treatment of hypercholesterolemia and obesity by reducing the cholesterol content in modern people at high risk of hypercholesterolemia and obesity.

In particular, since high levels of LDL-cholesterol (LDL-derived cholesterol, LDL-C) in plasma, referred to as hypercholesterolemia, is a significant risk factor for atherosclerotic cardiovascular disease (CVD), LDL-C has traditionally been considered as a therapeutic strategy for treating this disease. One of the therapeutic strategies is to use the pathway of regulating low-density lipoprotein receptor levels in the liver in order to treat hypercholesterolemia. Drug development has been conducted based on the action of removing LDL particles from the blood through induction of low-density lipoprotein receptor expression. With regard to this therapeutic strategy, a previous study has revealed that Platycodin D has an effect of lowering cholesterol in the body of hypercholesterolemia-induced rat (European Journal of Pharmacology (2006) 537; 166-173). However, an underlying molecular mechanism of the therapeutic effect of Platycodin D on hypercholesterolemia has not yet been elucidated, and it has not yet been demonstrated whether a synergistic effect occurs when administered in combination with existing chemotherapeutic agents.

In view of this technical background, the present inventors confirmed that when Platycodon grandiflorum or Platycodin D derived therefrom is administered in combination with an existing chemotherapeutic agent for hypercholesterolemia, low-density lipoprotein receptor (LDLR) expression in hepatocytes is induced to lower high cholesterol levels, and accordingly, based on a specific molecular mechanism thereof, the present invention has been completed.

### SUMMARY OF THE INVENTION

An object of the present disclosure is to provide a pharmaceutical composition for combined administration for preventing or treating hypercholesterolemia, the pharmaceutical composition including a Platycodon grandiflorum extract or Platycodin D derived therefrom, and a statin-class drug as active ingredients.

Another object of the present disclosure is to provide use of a composition in preventing or treating hypercholesterolemia, the composition including a Platycodon grandiflorum extract or Platycodin D derived therefrom, and a statin-class drug as active ingredients.

Still another object of the present disclosure is to provide an anti-hypercholesterolemia supplement agent including a Platycodon grandiflorum extract or Platycodin D derived therefrom as an active ingredient.

Still another object of the present disclosure is to provide a kit for preventing or treating hypercholesterolemia, the kit including a Platycodon grandiflorum extract or Platycodin D derived therefrom, and a statin-class drug as active ingredients.

Still another object of the present disclosure is to provide a method of preventing or treating hypercholesterolemia, the method including the step of administering, to an individual excluding humans, a Platycodon grandiflorum extract or Platycodin D derived therefrom, and a statin-class drug in combination.

Still another object of the present disclosure is to provide a composition for treating or preventing hypercholesterolemia, the composition including a Platycodon grandiflorum extract or Platycodin D derived therefrom as an active ingredient.

Still another object of the present disclosure is to provide a method of increasing expression of low-density lipoprotein receptor, the method including the step of treating hepatocytes in vitro with a Platycodon grandiflorum extract or Platycodin D derived therefrom.

### BRIEF DESCRIPTION OF THE DRAWINGS

FIGS. 1A and 1B show effects of PG and PD on viability of HepG2 cells; specifically. FIG. 1A shows results of treating HepG2 cells with PG at concentrations of 10 μg/mL, 50 μg/mL, 100 μg/mL, 250 μg/mL, and 500 μg/mL for 24 h, and FIG. 1B shows results of treating the cells with PD (1 μM, 2.5 μM, 5 μM) for 24 h, wherein the cell viability was measured using WST-8 analysis, and data is represented as mean±standard deviation (SD);

FIGS. 2A to 2C show results of PG and PD that induce LDLR expression and LDL uptake; specifically, FIG. 2A shows results of treating HepG2 cells with PG or PD for 24 h, wherein protein expression was examined by Western blot analysis. FIG. 2B shows results of treating HepG2 cells with PG and PD for 24 h, wherein cell surface LDLR levels were examined by flow cytometry, average fluorescence intensity of LDLR was shown as fold change, error bars are represented the mean t standard deviation, and *, P<0.05 represents the significant difference, and FIG. 2C shows results of treating HepG2 cells with PG (250 μg/mL) and PD (2.5 μM) for 24 h, followed by incubating with 5 μg/mL of BODYPI-FE-LDL for 1 h, wherein confocal microscopic images shows LDL (green) and DAPI-labeled nuclear staining (blue), quantification of LDL fluorescence intensity was analyzed using image J. Data, and data represents total LDL density per cells and the mean±standard deviation (SD), and *, P<0.05 represents the significant difference;

FIGS. 3A to 3D show that enhancement of LDLR protein stability by PG or PD is mediated by IDOL inhibition; specifically, FIG. 3A shows results of treating cells with PG (250 μg/mL) and PD (1 μM, 2.5 μM) for 24 h, in which LDLR mRNA expression levels were detected by RT-PCT, the bar graph represents fold change normalized to control using Image J, and the result represents the mean±standard deviation from three independent experiments, FIG. 3B shows results of Western blotting of cells after treating with PG or PD at indicated concentrations for 24 h, FIG. 3C shows results of detecting IDOL mRNA expression levels by RT-PCR, wherein *, P<0.05 represents the significant difference, and FIG. 3D shows results of treating cells with PD (2.5 μM) for 24 h, followed by treating with 100 μg/mL of CHX for the indicated time, wherein LDLR protein was analyzed by Western blotting, and data show quantification of LDLR intensity using Image J;

FIGS. 4A and 4B show that PD inhibits IDOL expression but does not inhibit LXRα expression; specifically, FIG. 4A shows results of co-treating HepG2 cells with PD (2.5 μM) and T0901317 (10 μM) for 24 h, wherein IDOL mRNA was measured by RT-PCR, and GAPDH was used as a loading control, and FIG. 4B shows results of treating cells with PG (250 μg/mL) or PD (1 μM, 2.5 μM, 5 μM) for 24 h, wherein LXRα expression was detected by Western blotting;

FIG. 5 shows that PG and PD exhibit a synergistic effect with simvastatin on LDLR expression, wherein HepG2 cells were co-treated with PG (250 μg/mL) or PD (2.5 μM) and simvastatin (1 μM) for 24 h, LDLR protein was detected by Western blotting, and quantification results represents the mean±standard deviation from three independent experiments, and *, P<0.05 represents the significant difference:

FIG. 6 shows that PG and PD exhibit a synergistic effect with simvastatin on LDL uptake, wherein HepG2 cells were co-treated with PG (250 μg/ml) or PD (2.5 μM) and simvastatin (1 μM) for 24 h, followed by incubating with 5 μg/mL of BODYPI-FE-LDL for 1 h, data represents the mean±standard deviation, and *, P<0.05 represents the significant difference; and

FIG. 7 shows a schematic model for the hypocholesterolemic action of PD through induction of LDL uptake in HepG2 cells, wherein PG and PD induce LDLR expression through regulation of protein stability caused by IDOL inhibition, and increases plasma LDL uptake levels in HepG2 cells, and co-treatment of PD or PG with simvastatin markedly enhances the cholesterol-lowering effect of simvastatin.

### DETAILED DESCRIPTION OF THE PREFERRED EMBODIMENTS

Each description and embodiment disclosed in this disclosure may also be applied to other descriptions and embodiments. That is, all combinations of various elements disclosed in this disclosure fall within the scope of the present disclosure. Further, the scope of the present disclosure is not limited by the specific description described below.

To achieve the above objects, an aspect of the present disclosure provides a pharmaceutical composition for combined administration for preventing or treating hypercholesterolemia, the pharmaceutical composition including a Platycodon grandiflorum extract or Platycodin D derived therefrom, and a statin-class drug as active ingredients.

Further, the present disclosure provides use of a composition in preventing or treating hypercholesterolemia, the composition including a Platycodon grandiflorum extract or Platycodin D derived therefrom, and a statin-class drug as active ingredients.

The Platycodon grandiflorum extract of the present disclosure or Platycodin D included therein is significant in that it is administered in combination with an existing chemotherapeutic agent for hypercholesterolemia to induce expression of hepatic low-density lipoprotein receptor (LDLR), thereby promoting the development of a prophylactic or therapeutic agent for hypercholesterolemia.

As used herein, the term “Platycodon grandiflorum” refers to a medicinal herb prepared by removing the root or periderm of the Platycodon grandiflorum A. De Candolle belonging to the family Campanulaceae. Platycodon grandiflorum is generally used as an herb medicine, and is used for cough, gallstones, expectorant, nasal congestion, cold, tonsillitis, and empyema in oriental medicine.

The Platycodon grandiflorum may be collected from nature, or may be cultivated, or may be purchased, and may be obtained by using other known methods. In addition, the Platycodon grandiflorum extract may be extracted from a natural, hybrid, or variant plant, and may be extracted from a plant tissue culture.

As used herein, the term “extract” includes a liquid extract itself and an extract of any formulation which may be prepared using the liquid extract, such as a liquid extract obtained by extraction treatment of Platycodon grandiflorum, a diluted or concentrated liquid of the liquid extract, a dried product obtained by drying the liquid extract, and a crude purification product or a purification product of the liquid extract, or a mixture thereof, etc.

The Platycodon grandiflorum extract may be obtained by extracting using a solvent selected from the group consisting of water, C₁ to C₄ alcohols, and mixed solvents thereof, and specifically, it may be a hot water extract, but is not limited thereto.

The extraction method is not particularly limited, and extraction may be performed at room temperature or by heating under conditions where the active ingredient is not destroyed or its destruction is minimized. Specifically, the method of obtaining the Platycodon grandiflorum extract in the present disclosure may be as follows. A polar solvent of water or C₁ to C₄ alcohols, such as methanol, ethanol, propanol, butanol, etc., in a volume of about 2 times to about 20 times, specifically about 3 times to 5 about times the weight of Platycodon grandiflorum, or a mixed solvent thereof at a mixing ratio of about 1:0.1 to about 1:10 may be used as an extraction solvent, but is not limited thereto. The extraction temperature may be 1° C. to 100° C., specifically 15° C. to 35° C., and the extraction period may be about 1 h to about 10 days, specifically 2 h to 50 h, and the extraction method may be shaking extraction, hot water extraction, cold immersion extraction, reflux cold extraction, ultrasonic extraction, or a combination thereof, but is not limited thereto.

The Platycodon grandiflorum extract may be 0.01% by weight to 99% by weight, specifically 0.05% by weight to 90% by weight, and more specifically 0.1% by weight to 80% by weight, based on the total composition, but is not limited thereto.

As used herein, the term “Platycodin D” is a compound represented by the following Chemical Formula 1, and has a chemical formula of C₅₇H₉₂O₂₈, and is a type of saponin.

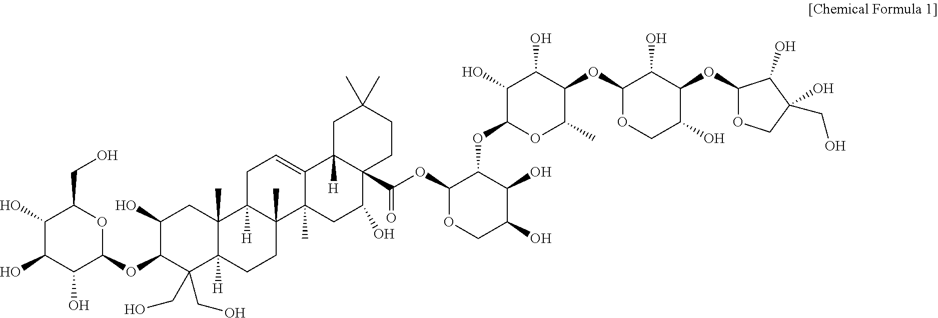

In the present disclosure, as the Platycodin D compound, a product extracted from a natural source may be used, but a product which is chemically synthesized may be used, as long as it exhibits the same effect as the extract from the natural source, but is not limited thereto.

The Platycodin D compound of the present disclosure may be specifically extracted and purified from Platycodon grandiflorum (Platycodi radix), but is not particularly limited thereto, and may be extracted from various natural sources other than Platycodon grandiflorum. The Platycodin D may be isolated and purified from the Platycodon grandiflorum extract by way of a common method known in the art, i.e., it may be isolated and purified using a common solvent under common temperature and pressure conditions.

The saponins are classified as triterpenes and steroids, based on aglycone attached to sugar moiety by glycosidic linkage. In addition, these saponin groups may be distinguished according to sapogenin, and among them, Platycodon grandiflorum saponin is a pentacyclic oleanane-type triterpene saponin. Specifically, Platycodon grandiflorum includes about 2% of about 10 kinds of triterpene saponins as saponins. Among these saponins, Platycodin A, C, D, and D2, and two kinds of monoacetates, Platycodin D3 were reported as glycosides of platycodigenin (Rossi M. et al., Chem. Pharm. Bull 16(11):2300, 1968).

As used herein, the term “statin-class drug”, which refers to a drug of the HMG-CoA reductase inhibitor family, means a chemotherapeutic agent used as a therapeutic agent for hypercholesterolemia, and specifically, the statin-class drug may be one or more selected from the group consisting of lovastatin, simvastatin, pravastatin, fluvastatin, atorvastatin, and cerivastatin, but is not limited thereto.

The HMG-CoA reductase inhibitor is mainly present in the microsomal fraction of hepatocytes. In a process of synthesizing cholesterol from acetic acid, the HMG-CoA reductase inhibitor is a rate-limiting enzyme that catalyzes a reaction of p-hydroxy-D-methylglutaryl CoA to mevalonic acid to regulate reactions in cholesterol synthesis metabolism. Therefore, the HMG-CoA reductase inhibitor is involved in the regulatory mechanism of cholesterol synthesis and is inhibited or promoted by various factors.

Specifically, two major enzymes in the regulation of cholesterol metabolism are HMG-CoA reductase (3-hydroxy-3-methylglutaryl coenzyme A. EC 1.1.1.34) and ACAT (acyl coenzyme A: cholesterol acyltransferase, EC 2.3.1.26), which are present in the liver tissue and influence cholesterol synthesis and storage amount thereof. HMG-CoA reductase is a principal rate-limiting enzyme regulating cholesterol synthesis in the liver, and ACAT is an enzyme responsible for esterification of free cholesterol in the small intestine, liver, and other tissues, and is involved in intestinal uptake of dietary cholesterol, secretion of very-low-density lipoprotein (VLDL)-cholesterol in the liver, and cholesterol accumulation in arterial vessels (Helgerud, 1981; Sucking & Stange, 1985). According to reports thus far, inhibition of these two enzymes not only exhibited cholesterol-lowering effects in various experimental animals including humans, but also exhibited anti-arteriosclerosis effects (Alberts et al., 1980; Amin et al., 1993; Alberts, 1988; Fujioka et al., 1995; Schnizer-Polokoff et al., 1991; Largis et. al.., 1989; Heidner et al., 1983; William et al., 1989; Roth et al., 1992; Sugiyami et al., 1995).

As used herein, the term “hypercholesterolemia” refers to a disease in which cholesterol in the blood exceeds the normal value, and may be used in the same sense as high blood cholesterol.

The cholesterol acts as a precursor of steroid substances. In particular, since sex hormones and adrenocortical hormones are steroid hormones, cholesterol is absolutely necessary. However, when a lot of lipoproteins accumulate in blood vessels due to obesity, etc., the blood cholesterol levels increase. Thus, when many lipoproteins accumulate in blood vessels due to obesity, etc., blood cholesterol levels may increase.

As used herein, the term “preventing” means all of the actions by which hypercholesterolemia is restrained or retarded by administering the Platycodon grandiflorum extract according to the present disclosure, the active ingredient derived therefrom, or the composition.

As used herein, the term “treating” means all of the actions by which symptoms of hypercholesterolemia have taken a turn for the better or been modified favorably by administering the Platycodon grandiflorum extract according to the present disclosure, the active ingredient derived therefrom, or the composition.

As used herein, the term “combined administration” means concurrent or sequential treatment.

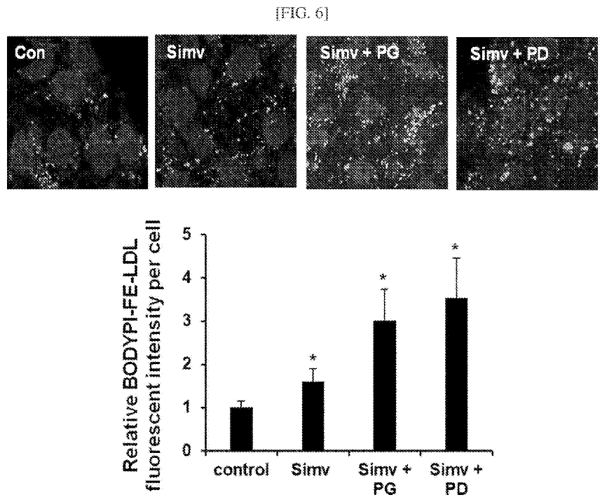

In a specific embodiment of the present disclosure, combined administration of the Platycodon grandiflorum extract or Platycodin D derived therefrom with a statin-class drug was found to exhibit the effect of lowering high levels of cholesterol, and in particular, the combined administration was found to remarkably increase the therapeutic effect (FIGS. 5 and 6).

In the present disclosure, the Platycodon grandiflorum extract or Platycodin D derived therefrom may be administered in combination with a statin-class drug to have a synergistic effect on the prevention or treatment of hypercholesterolemia, and in particular, the Platycodon grandiflorum extract or Platycodin D derived therefrom may be administered in combination with a statin-class drug to allow the statin-class drug to have an anti-hypercholesterolemia effect even in a small amount.

In the present disclosure, the pharmaceutical composition may increase expression of low-density lipoprotein receptor (LDLR).

The term “low-density lipoprotein receptor” refers to a low-density lipoprotein-binding cell surface in a metabolic process in which low-density lipoproteins bind to the cell surface of peripheral tissues to be taken into the cells. Therefore, cholesterol metabolism in peripheral tissues is regulated by uptake of low-density lipoprotein, and when abnormality is caused herein, cholesterol control action does not occur, resulting in excessive cholesterol production, leading to hypercholesterolemia.

Specifically, the term “low-density lipoprotein”, which is one of the fractions constituting plasma lipoprotein, means those produced as a result of the elimination of triglycerides of very-low-density lipoprotein (VLDL), etc. by enzymatic action.

In the present disclosure, the pharmaceutical composition may prevent or treat hypercholesterolemia by promoting uptake of LDL-cholesterol (LDL-derived cholesterol, LDL-C).

The LDL-cholesterol refers to cholesterol included in LDL, and when LDL cholesterol is 170 mg or higher, it is regarded as hyper-LDL-cholesterolemia.

In the present disclosure, the pharmaceutical composition may further include a pharmaceutically acceptable carrier, but is not particularly limited thereto.

The term “carrier” refers to a medium that provides an adhesion surface for microorganisms to proliferate and grow well inside a bioreactor.

The pharmaceutical composition according to the present disclosure includes the Platycodon grandiflorum extract or Platycodin D included therein as an active ingredient, and may further include a pharmaceutically acceptable carrier. The pharmaceutically acceptable carrier may be those commonly used for preparation, and includes, but is not limited to, saline, sterile water, Ringer's solution, buffered saline, cyclodextrin, a dextrose solution, a maltodextrin solution, glycerol, ethanol, liposomes, etc., and may further include another common additive such as an antioxidant, a buffer, etc. as needed. In addition, the pharmaceutically acceptable carrier may be prepared in injectable forms such as an aqueous solution, a suspension, an emulsion, etc., pills, capsules, granules, or tablets by further adding diluents, dispersants, surfactants, binders, lubricants, etc. Suitable pharmaceutically acceptable carriers and their preparations may be prepared according to each component using a method disclosed in the Remington literature reference. The pharmaceutical composition of the present disclosure is not particularly limited to dosage forms, and may be prepared as injections, inhalants, or topical formulations for skin. Another aspect of the present disclosure provides an anti-hypercholesterolemia supplement agent including the Platycodon grandiflorum extract or Platycodin D derived therefrom as an active ingredient.

Descriptions of the terms “Platycodon grandiflorum”. “extract”. “Platycodin D”, and “statin-class drug” are the same as described above.

As used herein, the term “anti-hypercholesterolemia supplement agent” refers to an agent that induces or aids to prevent, inhibit, or treat hypercholesterolemia.

In a specific embodiment of the present disclosure, combined administration of the Platycodon grandiflorum extract or Platycodin D derived therefrom with a statin-class drug was found to exhibit the effect of lowering high levels of cholesterol, and in particular, the combined administration was found to remarkably increase the therapeutic effect, indicating that the Platycodon grandiflorum extract or Platycodin D derived therefrom may be used as an anti-hypercholesterolemia supplement agent (FIGS. 5 and 6).

Still another aspect of the present disclosure provides a kit for preventing or treating hypercholesterolemia, the kit including the Platycodon grandiflorum extract or Platycodin D derived therefrom and a statin-class drug as active ingredients.

Descriptions of the terms “Platycodon grandiflorum”, “extract”, “Platycodin D”, “statin-class drug”, “hypercholesterolemia”, “preventing”, and “treating” are the same as described above.

As used herein, the term “kit” refers to a collection of parts that may be assembled to immediately make an object, and in the present disclosure, the kit means a collection of experimental supplies provided to prevent or treat hypercholesterolemia.

Still another aspect of the present disclosure provides a method of preventing or treating hypercholesterolemia, the method including the step of administering, to an individual, the Platycodon grandiflorum extract or Platycodin D derived therefrom, and a statin-class drug in combination.

Descriptions of the terms “Platycodon grandiflorum”, “extract”, “Platycodin D”, “statin-class drug”, “combined administration”, “hypercholesterolemia”. “preventing”, and “treating” are the same as described above.

As used herein, the term “administering” means introducing the composition including the Platycodon grandiflorum extract or Platycodin D derived therefrom and statin-class drug into an individual by any suitable method.

As used herein, the term “individual” means all animals of rats, mice, and livestock, including humans, which have developed or are at risk of developing hypercholesterolemia, but is not particularly limited thereto.

The pharmaceutical composition of the present disclosure may be administered in a pharmaceutically effective amount.

The term “pharmaceutically effective amount” means an amount which is sufficient to treat diseases at a reasonable benefit/risk ratio applicable to any medical treatment. The effective dosage level may be determined depending on factors including a kind of an individual and severity, age, sex, activity of a drug, sensitivity to a drug, administration time, administration route, excretion rate, duration of treatment, drugs used concurrently, and other factors known in the medical field.

The pharmaceutical composition may be administered alone or in combination with other therapeutic agents, and may be administered sequentially or simultaneously with existing therapeutic agents. The composition may be administered in a single- or multiple-dosage form. It is important to administer the composition in a minimum amount that may exhibit a maximum effect without causing side effects, considering all of the above-described factors. The amount may be readily determined by those skilled in the art.

Further, the pharmaceutical composition may be administered orally or parenterally (e.g., intravenous, subcutaneous, intraperitoneal or topical administration) depending on the purpose, and the administration dose may be properly selected by those skilled in the art, depending on a patient's conditions and body weight, severity of a disease, preparation of a drug, and administration route and time.

For specific example, the pharmaceutical composition may be generally administered in a daily dosage of 0.001 mg/kg to 1000 mg/kg, more specifically 0.05 mg/kg to 200 mg/kg, and most specifically 0.1 mg/kg to 100 mg/kg once per day or in several divided doses per day. A preferred dosage may be properly selected by those skilled in the art, depending on an individual's conditions and body weight, severity of a disease, preparation of a drug, and administration route and time.

Still another aspect of the present disclosure provides a composition for treating or preventing hypercholesterolemia, the composition including the Platycodon grandiflorum extract or Platycodin D derived therefrom as an active ingredient.

Descriptions of the terms “Platycodon grandiflorum”, “extract”, “Platycodin D”, “hypercholesterolemia”, “preventing”, and “treating” are the same as described above.

Still another aspect of the present disclosure provides a method of increasing expression of low-density lipoprotein receptor, the method including the step of treating hepatocytes in vitro with the Platycodon grandiflorum extract or Platycodin D derived therefrom.

Descriptions of the terms “Platycodon grandiflorum”, “extract”, “Platycodin D”, and “low-density lipoprotein receptor” are the same as described above.

The term “increasing expression” means increasing expression of mRNA or/and protein of low-density lipoprotein receptor in the present disclosure, but is not particularly limited thereto. Further, the increased expression may be measured by way of a known common method, specifically by Western blot analysis, but is not limited thereto.

Hereinafter, the present disclosure will be described in more detail with reference to the following exemplary embodiments. However, these exemplary embodiments are for illustrative purposes only, and the scope of the present disclosure is not intended to be limited by these exemplary embodiments.

### Example 1. Cell Culture and Chemicals

HepG2 cell line was purchased from the Korean Cell Line Bank (KCLB, Seoul, Korea). The cells were cultured in RPMI-1640 supplemented with 10% FBS and 5% antibiotic at 37° C. and 5% CO₂. Next, Platycodin D (PD) was purchased from Cayman Chemical Company (Ann Arbor, Mich., USA) and dissolved in DMSO at a concentration of 10 mM. In addition, Platycodon grandiflorum (PG) produced by Hanpoong Pharmaceutical Company (Jeonju) was dissolved at 20 mg/mL in distilled water.

### Example 2. Cell Viability Assay

Cells were dispensed in a 96-well plate and treated with PG (10 μg/mL, 50 μg/mL, 100 μg/mL, 250 μg/mL, 500 μg/mL) or PD (1 μM, 2.5 μM, 5 μM) for 24 h. After incubation, a WST solution (Daeillab Service, Co., Seoul, Korea) was added to each well and further incubated for 2 h. Next, water-soluble formazan in the medium was measured at 450 nm using an ELISA reader (Molecular Devices, Palo Alto, Calif.).

### Example 3. Western Blot Analysis

Cells were treated with PD and PG for 24 h. Proteins were separated on 10% SDS-PAGE and transferred to nitrocellulose membranes. The membranes were incubated overnight at 4° C. For reference, anti-LDLR, Mylip/IDOL and LXRα were purchased from Abcam (Cambridge, UK), and anti-GAPDH was purchased from Cell Signaling (MA, USA). Further, anti-PCSK9 was purchased from Proteintech (Chicago. USA). The blots were then incubated with HRP-conjugated secondary antibody for 1 h at room temperature and measured using a Pierce ECL Western Blotting Substrate (Thermo Scientific, MA, USA).

### Example 4. Reverse Transcription (RT)-PCR

Cells were dispensed in a 6-well plate, and treated with PD or PG for 24 h, and then total RNA was extracted using an R&A-BLUE Total RNA extraction kit (iNtRON Biotechnology, Inc., Korea). Next, complementary DNA (cDNA) was synthesized from 1 μg of RNA using a PrimeScript 1 st strand cDNA Synthesis Kit (Takara Biotechnology, Dalian. Liaoning, China). RT-PCR was performed with a Maxime PCR PreMix Kit (i-StarTaq) (iNtRON Biotechnology, Inc., Korea). Sequences of primers used herein are as in Table 1, and GAPDH was used as an internal control.

TABLE 1

| | Forward primer | Reverse primer |
|---|---|---|
| Gene | (5′-3′) | (5′-3′) |
| LDLR | CAGATATCATCAACGAAGC | CCTCTCACACCAGTTCACTCC |
| MYLIP | TTGTGGACCTCGTTTCAAGA | GCTGCAGTTCATGCTGCT |
| GAPDH | CGTCTTCACCACCATGGAGA | CGGCCATCACGCCACAGTTT |

### Example 5. LDL Uptake Assay

Cells were dispensed in a 24-well plate and treated as described above. After treatment with drugs, the culture medium was removed and replaced with 5 μg/mL of BODIPY-FL-LDL (Invitrogen, CA, USA) at 37° C. for 1 h. The cells were washed with PBS containing 0.3% BSA and fixed with 4% paraformaldehyde (PFA) for 10 min. Nuclei were stained with 1 μg/mL of DAPI in 2% BSA for 1 min and analyzed using confocal microscopy.

### Example 6. Cell Surface LDLR Analysis

Cells were treated as described above for 24 h, harvested, and then incubated at 37° C. for 30 min with anti-LDLR (Abcam, Cambridge, UK) diluted at 1:60 in PBS containing 5% BSA. The cells were washed with 1% BSA in PBS and incubated with Alexa Fluor 488-conjugated goat anti-rabbit IgG (Invitrogen, CA, USA) using a 1:250 dilution at 37° C. for 30 min. The cells were fixed with 0.5% PFA for 10 min, resuspended in PBS, and measured by flow cytometry (FACSCalibur, BD Biosciences, San Jose, Calif., USA). Data were analyzed using a CellQuest Pro software (BD Biosciences, USA).

### Example 7. Statistical Analysis

Data was presented as the mean±standard deviation (SD). Statistical analysis was performed using a student's t-test, p values<0.05 were considered to represent the significant difference.

### Experimental Result 1. Cytotoxic Effects of PG and PD on HepG2 Cells

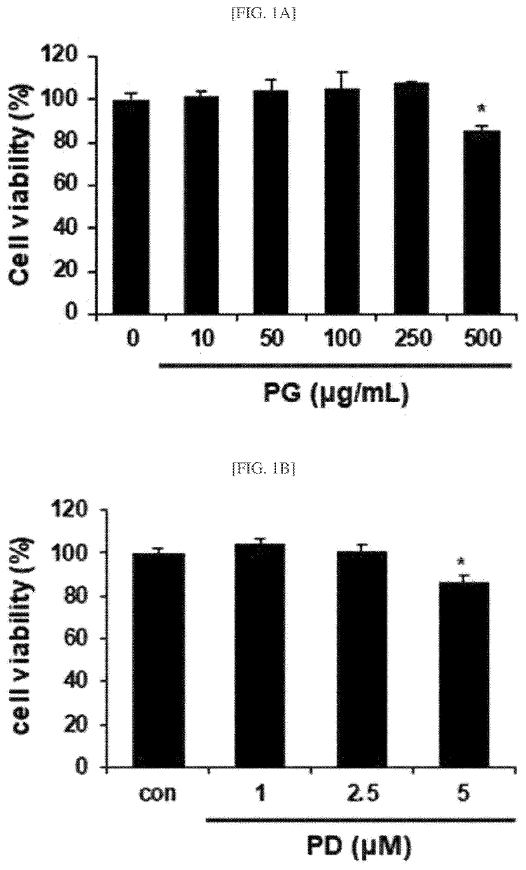

To investigate whether PG and PD display cytotoxic effects on HepG2 cells, cell viability was examined by WST1 assay following treatment with different concentrations of PG (10 μg/mL, 50 μg/mL, 100 g/mL, 250 μg/mL, 500 μg/mL) or PD (1 μM, 2.5 μM, 5 μM). As a result, as shown in FIGS. 1A and 1B. PG (10 μg/mL to 250 μg/mL) and PD (1 μM to 2.5 μM) had no significant cytotoxic effects on HepG2 cells. However, when high concentrations of 500 μg/mL of PG and 5 μM of PD were treated, cell viability was measured as 85% and 86%, respectively.

### Experimental Result 2. PD Inducing Cell Surface LDLR Expression and LDL Uptake in HepG2 Cells

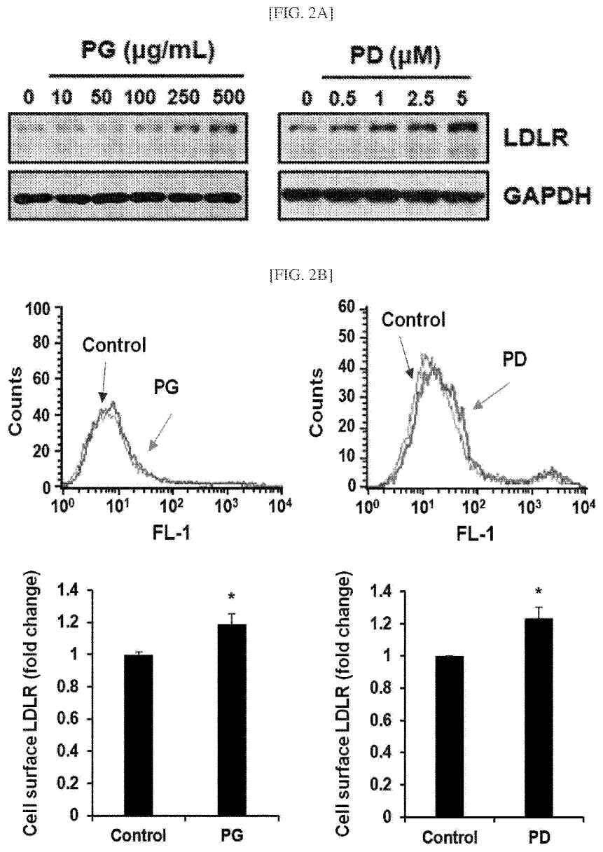

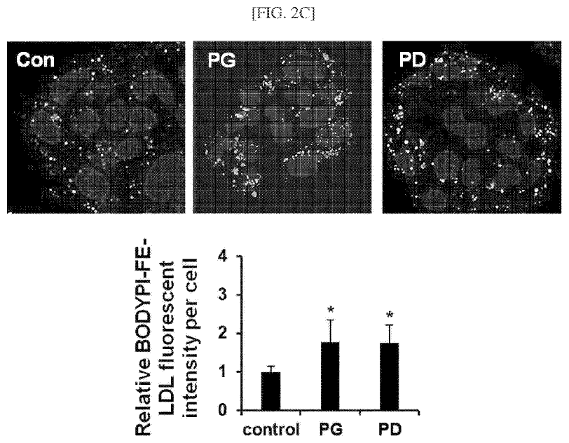

There is a report that LDLR mediates the uptake of LDL-induced cholesterol from plasma, lowering blood cholesterol levels. Therefore, it was examined whether PG and PD upregulate LDLR expression in HepG2 cells. As a result, as shown in FIG. 2A, Western blot analysis showed that PG and PD induce LDLR expression in HepG2 cells in a dose-dependent manner.

The highest levels of LDLR expression were observed at 500 μg/mL of PG and M of PD. However, the cytotoxic effect was observed at these concentrations, as confirmed in Experimental result 1. For the next experiments, 250 μg/mL of PG and 2.5 μM of PD were selected.

Next, FACS analysis was performed to examine whether PG and PD increase LDLR expression on cell surface. As a result, as shown in FIG. 2B, when 250 μg/mL of PG and 2.5 μM of PD were treated, cell surface LDLR levels were enhanced by 1.2±0.06 and 1.2±0.07, as compared with a control, respectively.

To further investigate whether PG and PD are able to increase the uptake of LDL-C, PG or PD-treated HepG2 cells were incubated with BODYPI-labeled LDL particles for 1 h. Then, confocal microscopy was performed. As a result, as shown in FIG. 2C. PG or PD-treated HepG2 cells showed a 1.7-fold increase in the uptake of LDL particles, as compared with untreated control cells.

### Experimental Result 3. PD Enhancing LDLR Protein Stability Through Down-Regulation of IDOL in HepG2 Cells

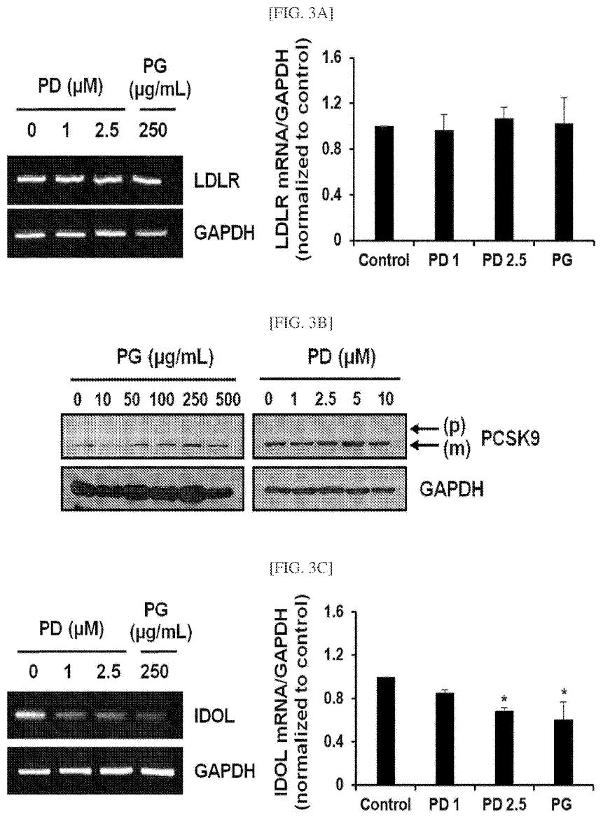

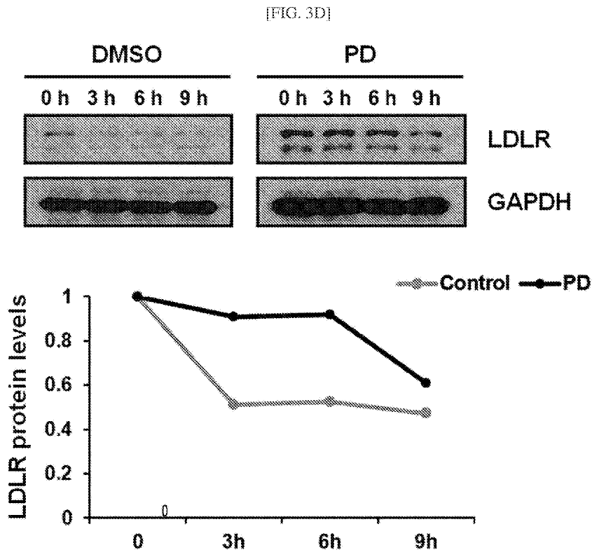

To define molecular mechanisms underlying the increased LDLR expression by PG and PD treatment, the effects of PG and PD on expression of genes including IDOL and PCSK9, which are known to regulate LDLR protein levels, were first examined. As a result, as shown in FIG. 3A, both PG and PD showed no significant effects on LDLR mRNA expression.

Further, as shown in FIG. 3B, it was confirmed that PG and PD do not affect a proteolytic process of PCSK9 that binds to LDLR and interferes with recycling back to the cell surface. However, as shown in FIG. 3C, both PG and PD significantly decreased mRNA levels of IDOL which is an E3 ubiquitin ligase that targets LDLR for its lysosomal degradation.

Considering that IDOL mRNA is decreased upon PG and PD treatment, it could be inferred that PG and PD are able to enhance LDLR protein stability. Therefore, LDLR protein levels remaining after treatment with cycloheximide (CHX) which blocks new protein synthesis were examined. As a result, as shown in FIG. 3D, LDLR proteins after treatment with CHX was found to be significantly stable in PD-treated cells, as compared with untreated cells.

### Experimental Result 4. LDLR Protein Stability According to Suppressed Mylip/IDOL Expression by PD

To examine whether PD suppresses IDOL mRNA expression, co-treatment of T0901317 (LXR agonist) and PD was performed for 24 h. The LXR is a transcription factor that activates a target gene involved in lipid and cholesterol homeostasis, and IDOL is a representative target gene of LXR. In addition. T0901317 increases the expression of IDOL by binding to LXR.

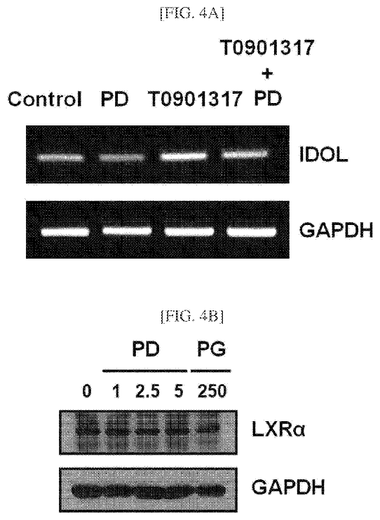

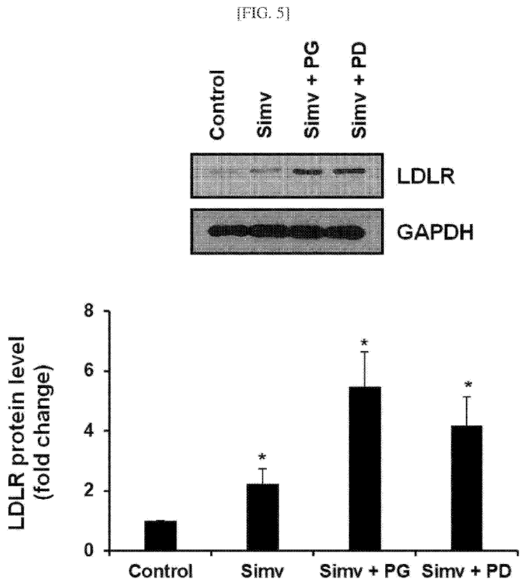

As a result, as shown in FIG. 4A, it was found that mRNA levels of IDOL gene were increased by T0901317, but this effect was attenuated by PD, indicating that PD inhibits the activated IDOL transcription.

According to these results, it was examined whether PD regulates LXR protein expression for IDOL inhibition. As a result, as shown in FIG. 4B, PD did not change LXR-α protein levels. These data indicate that PD contributes to LXR transcriptional activity, but may not contribute to protein expression.

### Experimental Result 5. PG and PD Exhibiting Synergistic Effect with Simvastatin on Hepatic LDLR Expression

Generally, statins are widely used for lowering blood levels of LDL-C, because they upregulate hepatic LDLR expression and enhance the uptake of LDL-C in the blood. To investigate whether PG and PD have a synergistic effect with statins on LDLR expression, HepG2 cells were treated with PG or PD together with simvastatin for 24 h, and then the expression levels of LDLR were examined by Western blotting.

As a result, it was found that simvastatin alone exhibited an approximately 2.2-fold increase in hepatic LDLR levels, as compared with untreated control cells. In particular, as shown in FIG. 5, the combined treatment of simvastatin with PG or PD synergistically increased LDLR levels by an average of 5.5- and 4.19-fold, as compared with control cells.

These results suggest that when PD or PG is administered in combination with the statin-class drug, hypercholesterolemia may be treated with the remarkably excellent synergistic effect.

### Experimental Result 6. PG and PD Exhibiting Synergistic Effect with Simvastatin on LDL Uptake

To investigate whether PG and PD have a synergistic effect with statins on LDL uptake, it was examined whether a synergistic increase in LDL uptake occurs according to increased LDLR expression upon combined administration with simvastatin. As a result, as shown in FIG. 6, combined administration of PG or PD with simvastatin increased the LDL uptake by around 3-fold and 3.5-fold, respectively.

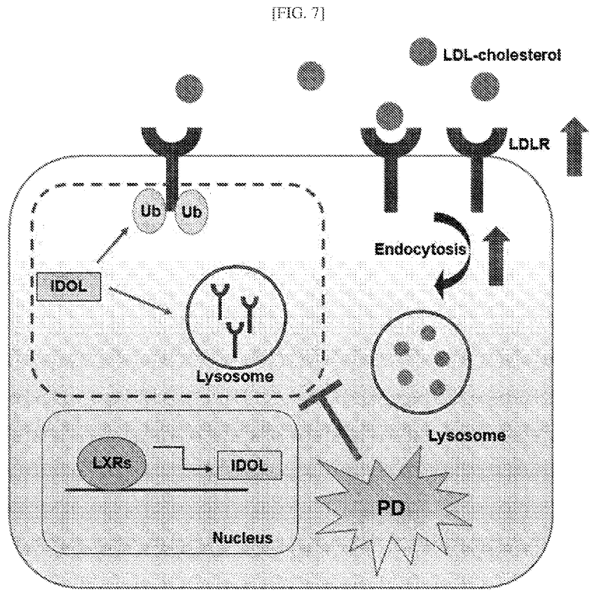

Taken together, as shown in FIG. 7, the present disclosure newly demonstrated that PG and PD induce LDLR expression and uptake of LDL-C particles by down-regulation of IDOL without affecting LXR expression. It was also confirmed that PG and PD exhibit a remarkably excellent synergistic effect with the statin-class drug on LDLR increase and LDL-C uptake, and therefore, they may be developed as a drug for remarkably lowering blood cholesterol levels.

Based on the above description, it will be understood by those skilled in the art that the present disclosure may be implemented in a different specific form without changing the technical spirit or essential characteristics thereof. In this regard, it should be understood that the above embodiment is not limitative, but illustrative in all aspects. The scope of the disclosure is defined by the appended claims rather than by the description preceding them, and therefore all changes and modifications that fall within metes and bounds of the claims, or equivalents of such metes and bounds, are therefore intended to be embraced by the claims.

### Effect of the Invention

A Platycodon grandiflorum extract of the present disclosure or Platycodin D included therein may be administered in combination with an existing chemotherapeutic agent for hypercholesterolemia to induce expression of hepatic low-density lipoprotein receptor (LDLR) to thereby effectively prevent or treat hypercholesterolemia.

1-10. (canceled)

11. A method of preventing or treating hypercholesterolemia, the method comprising the step of administering, to an individual, a Platycodon grandiflorum extract or Platycodin D derived therefrom, and a statin-class drug in combination.

12. (canceled)

13. A method of increasing expression of low-density lipoprotein receptor, the method comprising the step of treating hepatocytes in vitro with a Platycodon grandiflorum extract or Platycodin D derived therefrom.

14. The method of claim 11, wherein the Platycodon grandiflorum extract is extracted using a solvent selected from the group consisting of water, C₁ to C₂ alcohols, and mixed solvents thereof.

15. The method of claim 11, wherein the statin-class drug is one or more selected from the group consisting of lovastatin, simvastatin, pravastatin, fluvastatin, atorvastatin, and cerivastatin.

16. The method of claim 11, wherein the Platycodon grandiflorum extract or Platycodin D derived therefrom is administered in combination with the statin-class drug to have a synergistic effect on preventing or treating hypercholesterolemia.

17. The method of claim 11, wherein the composition increases expression of low-density lipoprotein receptor (LDLR).

18. The method of claim 11, wherein the composition promotes uptake of LDL-cholesterol (LDL-derived cholesterol, LDL-C).
